(19) United States
(12) Patent Application Publication
(10) Pub. No.: US 2012/0295362 A1
(43) Pub. Date: Nov. 22, 2012

(54) Urinalyisis test for synthetic canabanoids

(76) Inventor: Todd Allen Bland, Omaha, NE (US)

(21) Appl. No.: 13/135,609

(22) Filed: Jul. 12, 2011

Related U.S. Application Data
(60) Provisional application No. 61/400,706, filed on Aug. 2, 2010.

(51) Int. Cl. G01N 33/94; G01N 21/75

(52) U.S. Cl. 436/92; 422/417

(57) ABSTRACT

My newly created invention consists of a means to test Synthetic Cannabanoids, By a urinalysis cup which a new reagent patch is added to the other patches being tested for drugs such as Marijuana, cocaine, methamphetamines, opiates and Heroin but not limited to the said illegal drugs. An equivalent would be a Saliva test that gives immediate results but is more expensive, hence the UA cup is more economical. The Synthetic Cannabanoid drug stays in your blood stream for approximately three days, so the need to detect it is substantial as it has proven very harmful to users as it mimic's marijuana but can be far more potent then THC, The active ingredient in Marijuana.

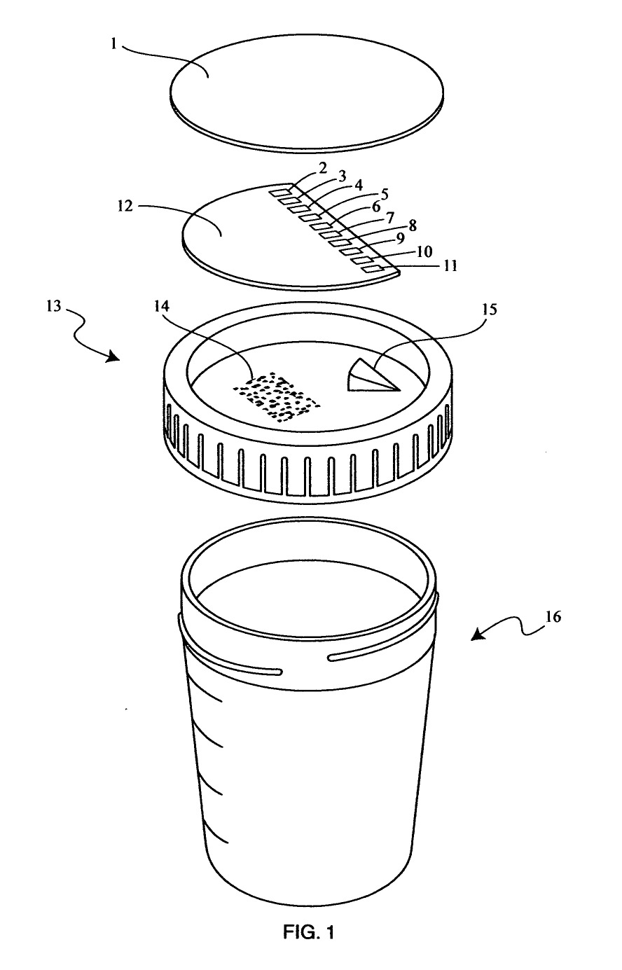

The parent drugs of the synthetic Cannabanoids are JWH-018, JWH-073, and JWH 250 respectively and not intended to limit them to these chemicals for the purpose of testing for Synthetic Cannabanoids.

The synthetic Cannabanoids are also snorted and it is sold in a powder form thereby the usage of this drug has caused a surge of Emergency room visits, seizures, difficulty in breathing, heart racing, hallucinations, paranoid behavior, agitation, nausea, vomiting, elevated blood pressure.

The study of JWH-018 are conjugates with glucronig acid and can reliably be tested and detected with GL-MS/MS and LC-MS-MS/MS. A reliable reagent test is the Marquis test and is accepted by the DEA.

### CROSS REFERENCE TO RELATED APPLICATIONS

This application claims the benefit of Provisional Patent Application 61/400,706 titled “Urinalysis Test for Synthetic Canabanoids, filed on Aug. 2, 2010 in United States Patent and trademark Office.

The specification of the above referenced patent application is incorporated herein by reference in it's entirety.

### BACKGROUND

Overtime Urinalysis tests have included new tests for specific Illegal drugs including Cocaine, Marijuana (THC), Methamphetamine's, Amphetamines, and Opiates.

Conventional detecting methods utilizing a conventional drug testing cup, such as Nugent, Edward U.S. Pat. No. 4,827,944 classification 600/584 or Davis U.S. Pat. No. 5,119,830, Attached is the SB08 with the two inventors patent information.

Have not included a very dangerous new drug referred to as Synthetic Canabanoids. Compounds' are changed frequently to avoid detection but the Marquis test for the said Synthetic Canabanoids has proven to be reliable as confirmed by the Drug Enforcement Agency.

The Synthetic Canabanoids are spiked onto leaf like substances Potpourri and the like or equivalent. The Synthetic canabanoid is also made into a powder which is more potent. The customers smoke or snort the product. The product is sold as incense but mimics' THC in marijuana.

The effects may be similar but have proven to be adversely affecting users such as difficulty in breathing, paranoia, seizures suicide, and hallucinations. Additionally, Increase in emergency room visits due to the Synthetic Canabanoids has been realized.

The top compounds currently illegal according to the DEA are JWH-018, CP-47,497, JWH-073, JWH-081 ,JWH-200, JWH-250, but are not limited to the above mentioned formulas.

The test is not intended to limit the testing of the Synthetic Canabanoids to the above mentioned Canabanoids but is intended to detect the Canabanoids of all equivalents.

The Marquis test for Synthetic Canabanoids adheres to the mandatory guidelines for Federal Drug testing.

My newly invented Synthetic Canabanoid Marquis test for Urinalysis specimens' or equivalent reagent that tests for all Synthetic Canabanoids of urine or equivalent reagent tests will meet mandatory guidelines for NIDA.

After the subject urinates into the cup using the Marquis test or equivalent, the administrator then turns the cup over and in approximately 5 minutes the results will appear changing the impregnated reagent patches colors such as shades of yellow or orange, but not limited to other colors according to the chemicals contained in the subjects urine.

After it is determined if the subject tests positive for the Synthetic Canabanoids the subjects sample can be verified with liquid chromatography, and mass spectrometry.

### SUMMARY OF THE INVENTION

Synthetic Canabanoid use has resulted in surges in emergency room Visits, calls to poison control centers, adverse effects but not limited to Seizures, hallucinations, paranoid behavior, agitation, nausea, vomiting, racing heart, and elevated blood pressure and even death. The need for a UA drug test for Synthetic Canabanoids is greatly needed.

The tests of modern day do not test for Synthetic Canabanoids, hence a reliable test will be very useful to probation offices and Law enforcement across the nation.

The test cups are equipped with impregnated patches, utilizing the Marquis test or equivalent. When the impregnated reagent patches are saturated with urine causes a reaction to turn the patch a cetain color to determine positive or negative.

### BRIEF DECRIPTION OF DRAWINGS

(13) Information sited from Davis U.S. Pat. No. 5,119,830 June, 1992 a conventional Urinalysis' cup, Including my numbering system for Davis drawing. Attached SB08 for your review of said US patent.

A Drawing of Davis's now conventional UA cup in his U.S.Pat. No. 5,119,830 June, 1992 is used for reference and for the purpose of showing my newly created invention of detecting Synthetic Canabanoids through my Marquis Reagent patch placed on the test strip or equivalent or such other reagent tests impregnating patches for the test strips deemed reliable by the DEA.

Davis's patent U.S. Pat. No. 5,119,830/June 1992 states, the inner partitions are formed as a one-piece lid main member (13) with most of the rest of the lid whereas the outer partition (14) is mounted to cover an indentation (14) into a top surface of the lid main member to define the test space indentations.

The shell when one presses down on (15) the valve, An intregal hinge is attached to the valve, breaks away from the the rest of the floor (the top of the inside lid) allowing the urine to saturate the chemical strip (12).

The new improvement of the herein invention is the conventional cup now can test urine reliably for all synthetic canabanoids safely with the Marquis test approved by the DEA.

Todd Allen Blands new improvement is the following Synthetic Canabanoids test using the Marquis reagent test for (2) CP47, (3) JWH-200, (4) JWH-73, (5) JWH-18, (6) CP 497, (7) Opiates, (8) Amphetamines, (9) THC, (10) Methamphetamine, and (11) Cocaine.

The test has been deemed reliable by the DEA but the invention is not intended to limit the above Synthetic Canabanoids as they are changing constantly. The Marquis test is preferable but all equivelents are resorted to falling under the appended claims forthcoming.

### PREFERED EMBODIENT OF DETAILED DESRCRIPTION

My newly invented method is made to test for all Synthetic Canabanoids using a person urine and the Marquis reagent test or equivalent.

Specifically, A device such as a conventional Drug Testing Urinalysis cup is now capable of testing urine with my newly invented method utilizing the conventional drug testing cups for the improvement of utilizing the DEA approved urine test for testing Synthetic Canabanoids called the Marqui reagent test or equivalent or others such as Mecke and Mandelline reagent test for synthetic canabanoids; deserve to be mentioned.

My device such as a conventional drug testing urine cup drug testing strip is allocated with the marqui reagent for the following compounds that are banned nation wide by the DEA, JWH-018, JWH-073, JWH-200, CP-47, CP-497.

A conventional drug testing cup such as Davis U.S. Pat. No. 5,119,830 US patent which tests for Cocaine, methamphetamines, opiates, THC Marijuana, Amphetimines is now improved by adding the Marquis impregnated patches detecting all Synthetic canabanoids by changing the urine to a yellow, orange. brown or green. Depending on Negative or positive.

The conventional UA cup of Davis U.S. Pat. No. 5,119,830 issued in 1992. The improvement of my new invention is the patches are impregnated with the Marqui reagent for the following compounds JWH-018, JWH-073, JWH-200, CP-47 and CP-497, But not limited to the above compounds as the chemicals change frequently to avoid prosecution.

The test strips as they are impregnated with the Marquis reagent that turn color if positive or negative, are protected inside tubes and chambers as not to infect other patches and the testing can use such test instead of waiting a prolonged period.

This process is specifically for detecting Synthetic Canabanoids by color patches or equivelent and is not intended to limit the above mentioned chemicals used to make Synthetic Canabanoids as they are changed frequently to change detection but the parent compounds are JWH-018, JWH-073, JWH-200, CP47 and CP-497 but not limited to the said mentioned compounds as they change frequently.

On Davis U.S. Pat. No. 5,119,830 conventional drug testing cup. I renumbered for the purpose of showing what is new (1) inner portion.

Marquis reagent patch CP-47 is (2), JWH-018 is number (2), JWH-73 is (3), JWH-18 is (4), CP497 is (6), Opiates is (7), Amphetimines is (8),

### PREFERRED EMBODIEMENT OF THE DETAILED DESCRIPTON

THC is (9), Methamphetamines is (10), and Cocaine is (11). The test strip (12) is covered with foil to protect the test strip. Number (13) is the lid to the cup. The indentatations is (14), the break away valve is (15). The bottom of the cup is (16).

What is claimed is as follows:

1. A conventional drug testing cup used for detecting Cocaine, Methamphetamines, Amphetamines, THC, and Opiates in the urine is improved by: My new method utilizing the Marquis reagent test for detecting Synthetic canabanoids specifically but not limited to JWH-018, JWH-073, JWH-081, JWH-200, JWH-250, AM 694, CP-47-497, And CP 55, 940.

2. All equivelent Synthetic canabanoids can be tested with the Marquis reagent test utilizing a persons urine. But not limited to Mecke and Mandelline or equivalents deemed reliable by the DEA.

3. A new or conventional UA drug testing cup or equivelent(s) that detect any or all Synthetic canabanoids in the urine.

4. The method of claim (2) a reliable test strip of said reliable reagents test may be immersed in the cup to specifically test for Synthetic canabanoids.
